(19) United States
(12) Patent Application Publication
(10) Pub. No.: US 2026/0115113 A1
(43) Pub. Date: Apr. 30, 2026

(54) DEODORANT SPRAY

(71) Applicant: Lume Deodorant, LLC, New York, NY (US)

(72) Inventors: Shannon Klingman, New York, NY (US); Stephanie Richmond, New York, NY (US); Rebecca Reynandez, New York, NY (US); Nikita Bhatnagar, New York, NY (US)

(73) Assignee: Lume Deodorant, LLC, New York, NY (US)

(21) Appl. No.: 18/930,552

(22) Filed: Oct. 29, 2024

(51) Int. Cl. A61K 8/365; A61K 8/04; A61K 8/19; A61K 8/34; A61K 8/49; A61Q 15/00

(52) U.S. Cl. CPC A61K 8/365; A61K 8/046; A61K 8/19; A61K 8/34; A61K 8/4953; A61Q 15/00; A61K 2800/30

(57) ABSTRACT

The present disclosure relates to a topical aerosol deodorant composition that contains water, an organic solvent, an alpha-hydroxycarboxylic acid, one or more skin conditioning agents, optionally one or more hydrocarbon carrier agents, optionally caffeine or a xanthine derivative that has vasoconstrictive effects and a propellant. The topical aerosol deodorant composition may be sprayed onto a human's external skin in need or odor control such as the user's axilla, pudendum, feet, chest, abdomen, thigh, groin, pelvis and buttock without irritation.

### FIELD OF THE INVENTION

The present invention relates to deodorant compositions in spray form, preferably aerosol spray, that provide good odor control. The deodorant compositions exhibit excellent cosmetics and aesthetics properties. The deodorant compositions may be applied to any external surface of a body to control odor at the site of application. Application sites include, but are not limited to, axilla, pudendum, feet, chest, abdomen, thigh, groin, pelvis and buttock. The present invention further relates to methods for preparing the deodorant compositions and methods of using the deodorant compositions.

### BACKGROUND

Aerosol antiperspirant and deodorant compositions are well known in the art. Examples of such compositions are described in U.S. Pat. Nos. 5,098,694, 5,911,977 and 5,977,850 and U.S. Patent Application Publication Nos. 2008/0213192, 2010/0056647, 2022/0387275, 2024/0122820 and 2024/0082126. Many of these compositions employs high amounts of alcohol that may cause irritation when applied, especially to a user's thigh, groin, pelvic and/or iliac region. Some of these known compositions also employ solid fillers such as talc, starches, silica, calcium carbonate, perlite and mica. These solid fillers can leave an unpleasant and undesirable chalky residue. Still further, some of these compositions employ film forming polymeric agents that can leave a tacky feel after application.

Topical compositions that are useful in controlling body odors by adjusting the skin's microbiome with the use of an alpha-hydroxycarboxylic acid such as mandelic acid are described in U.S. Pat. Nos. 9,668,948, 9,566,223, and U.S. Patent Application Publication No. 2022/0071878, which are incorporated herein by reference. Topical odor controlling products containing mandelic acid are also commercially available under the trademarks LUME® and MANDO®.

Although numerous antiperspirant and deodorants are described in the art, there always remains a need to develop an improved upon these compositions.

### SUMMARY OF THE INVENTION

The present invention is a topical aerosol deodorant composition comprising: (i) water, (ii) one or more organic solvents, preferably one or more C₁-C₆ alcohols; (iii) one or more alpha-hydroxycarboxylic acids; and (iv) one or more skin conditioning agents. The aerosol compositions can be packaged in a suitable container along with a propellant to dispense the aerosol composition onto to the user's designated outer skin area. The skin conditioning agents include emollients, humectants and/or moisturizers. The aerosol deodorant compositions may further comprise one or more cosmetically acceptable auxiliary substances include such as hydrocarbon carrier agents, preservative agents, antioxidants agents, pH adjusting agents, buffering agents, chelating agents, fragrance agents and combinations of the foregoing.

In certain embodiments, the topical aerosol deodorant composition comprises a base composition and a propellant. The base composition comprises: (i) about 40 wt % to about 80 wt % water; (ii) about 5 wt % to about 50 wt % of one or more organic solvents; (iii) about 1 wt % to about 10 wt % of one or more alpha-hydroxycarboxylic acids; and (iv) about 1 wt % to about 40 wt % of one or more skin conditioning agents. In certain aspects of this embodiment, the aerosol deodorant compositions may further comprise about 0.1 wt % to about 10 wt % of caffeine or a xanthine derivative that has vasoconstrictive effects. In certain aspects of this embodiment, the aerosol deodorant compositions are free or substantially free of: (a) solid fillers; (b) metal compounds; (c) silicones; (d) film forming polymeric agents; (c) phthalates; (f) sulfites; (g) parabens and (h) combinations of the forgoing.

In certain embodiments, the topical aerosol deodorant composition comprises a base composition and a propellant wherein the base composition comprises: (i) about 40 wt % to about 80 wt % water; (ii) about 5 wt % to about 50 wt % of one or more organic solvents; (iii) about 1 wt % to about 10 wt % of one or more alpha-hydroxycarboxylic acids; (iv) about 1 wt % to about 40 wt % of one or more skin conditioning agents; (v) about 0.01 wt % to about 20 wt % of one or more hydrocarbon carrier agents; and (vi) one or more additional cosmetically acceptable auxiliary substances selected from the group consisting of preservative agents, antioxidants agents, pH adjusting agents, buffering agents, chelating agents, fragrance agents and combinations of the foregoing. In certain aspects of this embodiment, the aerosol deodorant compositions may further comprise about 0.1 wt % to about 10 wt % of caffeine or a xanthine derivative that has vasoconstrictive effects. In certain aspects of this embodiment, the aerosol deodorant compositions are free or substantially free of: (a) solid fillers; (b) metal compounds; (c) silicones; (d) film forming polymeric agents; (e) phthalates; (f) sulfites; (g) parabens and (h) combinations or the forgoing.

The topical aerosol deodorant compositions may be applied to various skin areas of a human user or subject in need of odor control such as the axilla, pudendum, feet, chest, abdomen, thigh, groin, pelvis and buttock. The topical aerosol deodorant compositions may be applied to the desired skin area once or more times a day. Alternatively, the topical aerosol deodorant compositions may be applied once every 24 to 72 hours or any interval therefore. Preferably, the topical aerosol deodorant compositions are applied shortly after showering or bathing, i.e. between about 1 to 60 minutes after showering or bathing, to the dry desired skin area.

The topical aerosol deodorant compositions are prepared by preparing a base composition comprising: (i) water, (ii) one or more organic solvents, preferably one or more C₁-C₆ alcohols; (iii) one or more alpha-hydroxycarboxylic acids; (iv) one or more skin conditioning agents and (v) one of more cosmetically acceptable auxiliary substances such as hydrocarbon carrier agents, preservative agents, antioxidants agents, pH adjusting agents, buffering agents, chelating agents, fragrance agents and combinations of the foregoing. The base composition is then loaded into a suitable dispensing container, such as an aluminum can, and a propellant is added to the base composition in the dispensing container.

### DETAILED DESCRIPTION OF THE INVENTION

Before the present invention is further described, it is to be understood that this invention is not limited to the particular embodiments described. It is also to be understood that the terminology used herein is for describing particular embodiments only, and is not intended to be limiting.

It should be noted that as used herein, the singular forms “a,” “an,” and “the” include plural referents unless the context clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are encompassed within the invention, subject to any specifically excluded limit in the stated range. Where the stated range includes one or both of the limits, ranges excluding either or both of those included limits are also included in the invention.

As used herein the term “substantially free” means the composition contains 2 wt % or less of the stated ingredient, 1.5 wt % or less of the stated ingredient, 1 wt % or less of the stated ingredient, or 0.5 wt % of the stated ingredient depending upon the context of the description. The term “free of” means the composition is free of detectable amounts of the stated ingredient.

The term “weight %” and “wt %” are used interchangeably and unless otherwise stated are based on the total weight of the base aerosol deodorant composition, i.e. without the propellant, or the final aerosol deodorant composition, i.e., base composition and propellant.

The present invention is a topical aerosol deodorant composition comprising: (i) water, (ii) one or more organic solvents, preferably one or more C₁-C₆ alcohols; (iii) one or more alpha-hydroxycarboxylic acids; and (iv) one or more skin conditioning agents. The aerosol deodorant compositions may further comprise one of more cosmetically acceptable auxiliary substances include such as hydrocarbon carrier agents, preservative agents, antioxidants agents, pH adjusting agents, buffering agents, chelating agents, fragrance agents and combinations of the foregoing.

In certain aspects of the broadest embodiment, the topical aerosol deodorant composition comprises a base composition and a propellant. The base composition comprises: (i) about 40 wt % to about 80 wt % water, preferably about 45 wt % to about 75 wt % water and more preferably about 50 wt % to about 70 wt % water; (ii) about 5 wt % to about 50 wt % of one or more organic solvents, preferably about 10 wt % to about 40 wt % of one or more organic solvents and more preferably about 15 wt % to about 30 wt % of one or more organic solvents; (iii) about 1 wt % to about 10 wt % of one or more alpha-hydroxycarboxylic acids, preferably about 1.5 wt % to about 7.5 wt % of one or more alpha-hydroxycarboxylic acids and more preferably about 2.0 wt % to about 5 wt % of one or more alpha-hydroxycarboxylic acids; (iv) about 1 wt % to about 40 wt % of one or skin conditioning agents, preferably about 3 wt % to about 30 wt % of one or more skin conditioning agents and more preferably about 5 wt % to about 25 wt % of one or more skin conditioning agents; (v) about 0.5 wt % to about 35 wt % of one or more hydrocarbon carrier agents, preferably about 1 wt % to about 30 wt % of one or more hydrocarbon carrier agents and more preferably about 2.5 wt % to about 25 wt % of one or more hydrocarbon carrier agents; (vi) 0 wt % to about 10 wt % of caffeine or a xanthine derivative that has vasoconstrictive effects, preferably about 0.1 wt % to about 7.5 wt % of caffeine or a xanthine derivative that has vasoconstrictive effects and more preferably about 0.5 wt % to about 5 wt % of caffeine or a xanthine derivative that has vasoconstrictive effects; (vii) one or more skin conditioning agents; and (viii) one or more additional cosmetically acceptable auxiliary substances selected from the group consisting of preservative agents, antioxidants agents, pH adjusting agents, buffering agents, chelating agents, fragrance agents and combinations of the foregoing. In certain aspects of this embodiment, the final aerosol deodorant composition will further comprise at one or more fragrance agents to mask or improve the odor of the aerosol deodorant composition during application and one or more preservatives. The fragrance agents and preservatives may be incorporated into the base composition prior to adding the base composition to the dispensing container or added to the dispensing container prior to, concurrently with or subsequent to the addition of the base composition to the dispensing container.

Some embodiments of the foregoing topical aerosol deodorant compositions, the base composition may also comprise about 1 wt % to about 10 wt %, preferably about 1.5 wt % to about 8 wt %, and more preferably about 2 wt % to about 5 wt %, of one or more solid fillers such as talc, starches, silica, calcium carbonate, perlite and mica.

Some embodiments of the foregoing topical aerosol deodorant compositions are free or substantially free of solid fillers such as talc, starches, silica, calcium carbonate, perlite and mica.

Some embodiments of the foregoing topical aerosol deodorant compositions, the base composition may also comprise about 5 wt % to about 30 wt %, preferably about 10 wt % to about 25 wt %, and more preferably about 15 wt % to about 20 wt %, of one or more metal compounds such as aluminum, zirconium and zinc antiperspirant compounds such as those described in in U.S. Patent Application Publication No. 2013/0280175 and incorporated herein by reference and/or an odor suppressing compound such as sodium bicarbonate.

Some embodiments of the foregoing topical aerosol deodorant compositions are free or substantially free of metal compounds including but not aluminum, zirconium and zinc antiperspirant compounds such as those described in in U.S. Patent Application Publication No. 2013/0280175 and odor suppressing compounds such as sodium bicarbonate.

Some embodiments of the foregoing topical aerosol deodorant compositions are free or substantially free of silicones and more preferably free of cyclic and polymeric silicones.

Some embodiments of the foregoing topical aerosol deodorant compositions are free or substantially free of film forming polymeric agents. As used herein, film forming polymeric agents are polymers that are soluble in one or more of the materials employed in the topical aerosol deodorant compositions such as the organic solvents or carrier agents and after evaporation of the solubilizing material form a polymeric film.

Some embodiments of the foregoing topical aerosol deodorant compositions are free or substantially free of phthalate compounds.

Some embodiments of the foregoing topical aerosol deodorant compositions are free or substantially free of sulfite compounds.

Some embodiments of the foregoing topical aerosol deodorant compositions are free or substantially free of parabens compounds.

The topical aerosol deodorant base composition should exhibit a pH of about 2, 2.5, 3, 3.5, 4, 4.5, 5, 5.5, 6, 6.5, 7 or any range within the aforementioned values. The pH may be determined by any means commonly used in the industry including the use of a pH meter or commercially available pH paper, examples of which are provided in U.S. Patent Application Publication No. 2022/0071878, which is incorporated herein by reference.

The one or more organic solvents are organic compounds that are a liquid at room temperature and standard atmospheric pressure and preferably have a boiling point less than 200° C. Examples of organic solvents include but are not limited to methanol, ethanol, propanol, isopropanol, methyl acetate, t-butyl acetate, benzyl alcohol, cyclohexanol, glycerin, propylene glycol, butylene glycol, hexylene glycol and combinations of the foregoing. The preferred organic solvents are C₁-C₆ alcohols, and more preferably to methanol, ethanol, propanol, isopropanol and combinations thereof. In certain embodiments, the organic solvent comprises denatured alcohols, such as described in U.S. Pat. No. 5,098,694 or 5,997,850 and U.S. Patent Application Publication No. 2004/0136936, which are incorporated herein by reference.

The amount of the one or more organic solvents present in the topical aerosol deodorant base composition and or the final composition aerosol deodorant compositions can be about 5 wt %, 5.5 wt %, 6 wt %, 6.5 wt %, 7 wt %, 7.5 wt %, 8 wt %, 8.5 wt %, 9 wt %, 9.5 wt %, 10 wt %, 10.5 wt %, 11 wt %, 11.5 wt %, 12 wt %, 12.5 wt %, 13 wt %, 13.5 wt %, 14 wt %, 14.5 wt %, 15 wt %, 15.5 wt %, 16 wt %, 16.5 wt %, 17 wt %, 17.5 wt %, 18 wt %, 18.5 wt %, 19 wt %, 19.5 wt %, 20 wt %, 20.5 wt %, 21 wt %, 21.5 wt %, 22 wt %, 22.5 wt %, 23 wt %, 23.5 wt %, 24 wt %, 24.5 wt %, 25 wt %, 25.5 wt %, 26 wt %, 26.5 wt %, 27 wt %, 27.5 wt %, 28 wt %, 28.5 wt %, 29 wt %, 29.5 wt %, 30 wt %, 30.5 wt %, 31 wt %, 31.5 wt %, 32 wt %, 32.5 wt %, 33 wt %, 33.5 wt %, 34 wt %, 34.5 wt %, 35 wt %, 35.5 wt %, 36 wt %, 36.5 wt %, 37 wt %, 37.5 wt %, 38 wt %, 38.5 wt %, 39 wt %, 39.5 wt %, 40 wt %, 40.5 wt %, 41 wt %, 41.5 wt %, 42 wt %, 42.5 wt %, 43 wt %, 43.5 wt %, 44 wt %, 44.5 wt %, 45 wt %, 45.5 wt %, 46 wt %, 46.5 wt %, 47 wt %, 47.5 wt %, 48 wt %, 48.5 wt %, 49 wt %, 49.5 wt %, 50 wt % or any range encompassed by the forgoing values. The amount of the organic solvent present in the topical aerosol deodorant compositions should be kept below about 50 wt % to avoid irritation when the composition is applied especially to the thigh, groin or pelvic area.

Examples of the one or more alpha-hydroxycarboxylic acids that may be used in the topical aerosol deodorant compositions of the present invention include, but are not limited to, glycolic acid, mandelic acid, lactic acid, citric acid and combinations thereof. The amount of the one or more alpha-hydroxycarboxylic acids present in the topical aerosol deodorant composition base or final composition can be about 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt % or any range encompassed by the forgoing values.

Skin condition agents are compounds such as emollients, humectants, moisturizers, and the like, which improve the texture and feel of a composition when applied to a user's skin. Skin conditioning agents are known in the art and many can be found in typical reference textbooks such as the CTFA Cosmetic Ingredient Handbook or the International Cosmetic Ingredient Dictionary, published by the Cosmetic, Toiletry, and Fragrance Association, Washington D.C.

Emollient are typically an oleaginous or oily substance, which helps to smooth and soften the skin, and may also reduce its roughness, cracking or irritation. Some examples of emollients include mineral oil having a viscosity in the range of 50 to 500 centipoise (cps), lanolin oil, coconut oil, cocoa butter, olive oil, almond oil, macadamia nut oil, aloe extracts such as aloe vera lipoquinone, synthetic jojoba oils, natural sonora jojoba oils, safflower oil, corn oil, liquid lanolin, cottonseed oil and peanut oil. Further examples include a coco glyceride, which is a mixture of mono, di and triglycerides of cocoa oil, dicaprylyl ether, squalane, castor oil, polybutene, avocado oil, calophyllum oil, ricin oil, vitamin E acetate, silicone oils such as dimethylpolysiloxane and cyclomethicone, linotenic alcohol, oleyl alcohol, the oil of cereal germs such as the oil of wheat germ, isopropyl palmitate, octyl palmitate, isopropyl myristate, hexadecyl stearate, butyl stearate, decyl oleate, acetyl glycerides, the octanoates and benzoates of (C₁₂-C₁₅) alcohols, the octanoates and decanoates of alcohols and polyalcohols such as those of glycol and glyceryl, ricinoleates esters such as isopropyl adipate, hexyl laurate and octyl dodecanoate, dicaprylyl maleate, hydrogenated vegetable oil, phenyltrimethicone and combinations of the foregoing.

A humectant is a moistening agent that promotes retention of water due to its hygroscopic properties. Examples of humectants include glycerin, polymeric glycols such as polyethylene glycol and polypropylene glycol, mannitol and sorbitol.

Additional examples of skin conditioning agents can be found in U.S. Pat. Nos. 5,804,203 and 8,741,357, which are incorporated herein by reference.

Preferably, the one or more skin conditioning agents is a liquid at room temperature and atmospheric pressure. More preferably the one or more skin conditions agents are selected from vegetable oils, mineral oils, triglycerides, fatty acids, fatty alcohols, fatty acid esters and combinations thereof.

The amount of the one or more skin condition agents present in the topical aerosol deodorant base composition and or the final composition can be about 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt % or any range encompassed by the forgoing values.

The hydrocarbon carrier agents as used herein refers to compounds that consist solely of carbon and hydrogen atoms. The hydrocarbon carrier agents may be saturated or unsaturated hydrocarbons. The hydrocarbon carrier agents contributes to the odor stability of the topical aerosol deodorant composition i.e. prevents the development of malodor during storage of the topical aerosol deodorant composition. The hydrocarbon carrier agents also aids maintaining the homogeneity of the topical aerosol deodorant composition during storage of the composition, dispensing and application of the composition.

The hydrocarbon carrier agent is preferably a hydrocarbon that is a liquid at 20° C. and has at least six carbon atoms. Even more preferably, the hydrocarbon carrier agent is a saturated or unsaturated hydrocarbon with 6 to 20, preferably 8 to 18 and more preferably 10 to 16 carbon atoms and most preferably a saturated or unsaturated linear, i.e., non-cyclic hydrocarbon, with 10 carbon to 14 carbon atoms. Examples of hydrocarbon carrier agents include but are not limited to undecane, tridecane and mixtures thereof. In certain aspects the hydrocarbon carrier agent comprises a mixture of undecane and tridecane wherein the ratio of undecane to tridecane can range from 10:90, 20:80, 30:70, 40:60 50:50, 60:40, 70:30, 80:20, 90:10 or any range encompassed by the forgoing values.

The amount of the one or more hydrocarbon carrier agents present in the topical aerosol deodorant base composition and or the final composition can be about 0.01 wt %, 0.05 wt %, 0.1 wt %, 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt %, 10.5 wt %, 11 wt %, 11.5 wt %, 12 wt %, 12.5 wt %, 13 wt %, 13.5 wt %, 14 wt %, 14.5 wt %, 15 wt %, 15.5 wt %, 16 wt %, 16.5 wt %, 17 wt %, 17.5 wt %, 18 wt %, 18.5 wt %, 19 wt %, 19.5 wt %, 20 wt % or any range encompassed by the forgoing values.

In certain embodiments, the topical aerosol deodorant base composition and or the final composition of the present invention may also comprise about 0.1 wt %, 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt % or any range encompassed by the forgoing values of caffeine or xanthine derivatives that have vasoconstrictive effects. Examples of the xanthine derivatives are described in U.S. Patent Application Publication No. 2022/0071878, which is incorporated herein by reference.

Examples of preservatives that may be included in the topical aerosol deodorant base composition and or the final composition of the present invention include, but are not limited to, alkali metal sorbates, alkali metal benzoates, alcohols, glycols, parabens, quaternary-nitrogen containing compounds, isothiazolinones, aldehyde-releasing compounds and halogenated compounds. Specific examples of preservatives include phenoxyethanol, sodium benzoate, potassium benzoate, isopropyl alcohol, benzyl alcohol, sorbic acid and combinations thereof. The one or more preservatives are present in the topical aerosol deodorant compositions of the present invention in an amount of about 0.001 wt % to about 5 wt %, preferably about 0.0025 wt % to about 3 wt % and more preferably about 0.005 wt % to about 2.5 wt %.

Examples of antioxidants that may be employed in the topical aerosol deodorant base composition and or the final composition include, but are not limited to, niacinamide, peptides, such as di-, tri-, tetra and hexa-peptides, acetyl cysteine, ascorbic acid polypeptide, ascorbyl dipalmitate, ascorbyl methylsilanol pectinate, ascorbyl palmitate, ascorbyl stearate, butylated hydroxyanisole (BHA), butylated hydroxytoluene (BHT), t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, Helianthus annus (sunflower) seed oil, hydroquinone, isooctyl thioglycolate, kojic acid, laminaria digitata extract, magnesium ascorbate, magnesium ascorbyl phosphate, methylsilanol ascorbate, natural botanical antioxidants such as Camellia sinnensis green tea extract, Nelumbo nucifera leaf, flower or germ extract, vitris vinifera (grape) callous culture extract, nordihydroguaiaretic acid, octyl gallate, phenylthioglycolic acid, potassium ascorbyl tocopheryl phosphate, potassium sulfite, propyl gallate, quinones, rosmarinic acid, rosa mochata seed oil, Rosmarinus officinalis (rosemary) leaf extract, sodium ascorbyl phosphate, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, tremella fuciformis (mushroom) extract, tris(nonylphenyl)phosphite, Zea mays (corn) oil and combinations thereof.

The one or more antioxidants are present in the topical aerosol deodorant base composition and or the final composition of the present invention in an amount of about 0.001 wt % to about 2.5 wt %, preferably about 0.0025 wt % to about 2 wt % and more preferably about 0.005 wt % to about 1.5 wt %.

Examples of pH modifying agents that may be used in the topical aerosol deodorant base composition and or the final composition of the present invention include, but are not limited to, compounds that are added to raise or low the pH of the final composition as well as compounds that are added to buffer the topical aerosol deodorant base composition and or the final composition of the present invention. Compounds that may be added to raise or lower the pH of the topical aerosol deodorant base composition and or the final composition of the present invention include, but are not limited to, phosphoric acid and/or phosphate salts, citric acid and/or citrate salts, hydroxide salts (i.e., calcium hydroxide, sodium hydroxide, potassium hydroxide) and amines, such as triethanolamine.

Examples of buffer agents that may be used in the topical aerosol deodorant base composition and or the final composition of the present invention include, but are not limited to, acetic acid and salts, citric acid and salts, boric acid and salts, and phosphoric acid and salts such as mono- and di-potassium phosphate, citric acid/sodium citrate, and dibasic sodium phosphate/citric acid.

Examples of chelating agents that may be used in the topical aerosol deodorant base composition and or the final composition of the present invention include, but are not limited to, citric acid and salts thereof, disodium EDTA, tetrasodium EDTA, tetrasodium glutamate diacetate and phytic acid.

Examples of fragrances that may be used in the topical aerosol deodorant base composition and or the final composition of the present invention include, but are not limited to, oils such as angelica radix oil, aniseed oil, arnica blossom oil, basil oil, bay oil, bergamot oil, champak blossom oil, fir oil, turpentine oil, elemi oil, eucalyptus oil, fennel oil, pine needle oil, galbanum oil, geranium oil, ginger grass oil, guaiac wood oil, balsam of gurjun oil, Helichrysum/chasteweed oil, ho oil, ginger oil, iris oil, cajeput oil, calmus oil, chamomile oil, camphor oil, canaga oil, cardamom oil, cassia oil, fir cone oil, balsam of kopaiva oil, coriander oil, crisped mint oil, caraway oil, cumin oil, lavender oil, lemon grass oil, lime oil, tangerine oil, melissa oil, ambrette oil, myrrh oil, carnation oil, neroli oil, niaouli oil, oliban oil, orange oil, origanum oil, palmarosa oil, patchouli oil, peru balsam oil, petit grain oil, peppermint oil, pimento oil, pine oil, rose oil, rosemary oil, sandal wood oil, celery oil, lavender oil, star anise oil, turpentine oil, thuja oil, thyme oil, verbena oil, vetiver oil, juniper berry oil, absinthe oil, winter green oil, ylang-ylang oil, hyssop oil, cinnamon oil, cinnamon leaf oil, citronella oil, lemon oil, cypress oil and combinations thereof. Additional examples can be found in U.S. Patent Application Publication Nos. 2003/0138389 and 2010/0047296, which are incorporated herein by reference.

In addition to providing a user-friendly aroma to the topical aerosol deodorant composition, including masking any malodor provided by any individual components of the topical aerosol composition, the fragrance may also contribute to the odor stability of the topical aerosol deodorant composition, i.e. prevent the development of malodour during storage of the topical aerosol deodorant composition. In certain embodiments, the topical aerosol deodorant base composition and/or the final composition of the present invention will comprise 0 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 1.1 wt %, 1.2 wt %, 1.3 wt %, 1.4 wt %, 1.5 wt %, 1.6 wt %, 1.7 wt %, 1.8 wt %, 1.9 wt %, 2 wt %, 2.1 wt %, 2.2 wt %, 2.3 wt %, 2.4 wt %, 2.5 wt %, 2.6 wt %, 2.7 wt %, 2.8 wt %, 2.9 wt %, 3 wt % or any range encompassed by the forgoing values of one or more fragrance agents. Preferably the one or more fragrance agents comprises a citronella compound such as citronellyl methycrotonate, citronellyl acetate or combination thereof to provide an acceptable odor to the final topical aerosol deodorant composition during storage, dispensing and application. Additional examples of citronella compounds that may be used alone or in combination can be found in U.S. Patent Application Publication Nos. 2023/0015204 and 2024/0043770, which are incorporated herein by reference.

Additional specific examples of the cosmetically acceptable auxiliaries that may be used in the topical aerosol deodorant compositions of the present invention can be found the various U.S. Published patent applications mentioned previously as well as the CTFA International Cosmetic Ingredient Dictionary and Handbook (2008), 12^{th} Edition. The skilled artisan is aware that some of the cosmetically acceptable auxiliaries may be included in more than one of the foregoing classifications. Stated another way, some of the cosmetically acceptable auxiliaries may impart one or more properties to the topical aerosol deodorant composition.

Examples of propellants that may be used in the present invention include but are not limited to compressed air, nitrogen, inert Nobel gases such a helium and argon, carbon dioxide, nitrous oxide, and combinations thereof. Propellants may also include gaseous hydrocarbons like propane, n-butane, isobutene, cyclopropane, and mixtures thereof. Propellants my also include hydrofluorocarbons (HFCs), chlorofluorocarbons (CFCs). Additional examples of propellants can be found in U.S. Patent Application Publication Nos. 2008/0213192, 2014/0170102 and 2024/0122820, which are incorporated herein by reference. The propellant maybe present in the final topical aerosol deodorant composition at concentration from about 0.5 wt %, 1 wt %, 2 wt %, 3 wt %, 4 wt %, 5 wt %, 6 wt %, 7 wt %, 8, wt %, 9 wt %, 10 wt %, 11 wt %, 12, wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt % 50 wt % or any range encompassed by the forgoing values.

The propellant used in the topical aerosol deodorant compositions may be any suitable gas, or combination of gasses, that can be compressed or liquefied within a dispensing spray canister, which expand or volatilize to vapor or gas form upon exposure to ambient temperature and pressure conditions to deliver the topical aerosol deodorant composition in an aerosol form. The dispenser is commonly known in the art such as an aluminum can or glass bottle. Examples of suitable dispensers can be found in U.S. Pat. Nos. 4,396,152, 4,680,173, 5,082,652, and 11,059,659 and U.S. Patent Application Publication No. 2011/0127300, 2022/0143629, 2023/0015204 and 2024/0122820, which are incorporated herein by reference.

The topical aerosol deodorant composition is prepared by preparing a base composition comprising: (i) water, (ii) one or more organic solvents, preferably one or more C₁-C₆ alcohols; (iii) one or more alpha-hydroxycarboxylic acids; (iv) one or more skin conditioning agents and (v) one of more cosmetically acceptable auxiliary substances such as hydrocarbon carrier agents, preservative agents, antioxidants agents, pH adjusting agents, buffering agents, chelating agents, fragrance agents and combinations of the foregoing. The base composition is then loaded into a suitable dispensing container, such as an aluminum can, and a propellant is added to the base composition in the dispensing container.

The topical aerosol deodorant compositions of the present invention may be packaged and dispensed in any suitable container. Containers may include indicia on its surface that instruct the subject on its application and use. Instructions for use may also be printed separately and packaged with the container comprising the topical aerosol deodorant.

In certain embodiments, the user or human subject in need of or desiring odor control will apply, i.e. spray, the desired amount of the topical aerosol deodorant composition to a clean and preferably dry skin surface once every 6 to 72 hours or any interval therefore. Preferably, the topical aerosol deodorant composition is applied every 12 hours, 24 hours, 36 hours, 48 hours, 60 hours or 72 hours. The topical aerosol deodorant composition may be applied to various skin areas of a human subject in need or odor control such as the axilla, pudendum, feet, chest, abdomen, thigh, groin, pelvis and buttock. Preferably, the topical aerosol deodorant composition is applied shortly after showering or bathing, i.e. between about 1 to 60 minutes after showering or bathing, to the dry desired skin area. In certain embodiments, the user or subject will apply about a 1 second to 5 second spray to the target area. The amount will be adjusted depending upon the surface area to be treated.

Although any methods and materials similar or equivalent to those described herein can also be used in the practice of the present invention, the preferred methods and materials are now described. All publications mentioned herein are incorporated herein by reference to disclose and describe the methods and/or materials in connection with which the publications are cited.

The following examples are provided by way of illustration only and are by no means intended to be limiting.

### Example 1

A topical aerosol deodorant base composition, i.e. without the propellant, in accordance with the present invention may be prepared having the following:

| | WEIGHT PERCENT | | |
|---|---|---|---|
| | | More | Most |
| INGREDIENT | Preferred | Preferred | Preferred |
| Water | 40-80 | 45-75 | 50-70 |
| One or more C₁-C₆ alcohols | 5-50 | 10-40 | 15-30 |
| Mandelic acid, lactic acid or a | 1-10 | 1.5-7.5 | 2-5 |
| combination thereof | | | |
| One or more skin conditioning | 1-40 | 3-30 | 5-25 |
| agents | | | |
| One or more C₆-C₂₀ saturated or | 0.5-35 | 1-30 | 2.5-25 |
| unsaturated hydrocarbon carrier | | | |
| agents | | | |
| Caffeine | 0-10 | 0.1-7.5 | 0.002-2.5 |
| One or more preservatives | 0-5 | 0.001-3 | 1-9 |
| One or more fragrances | 0-3 | 0.1-2.5 | 0.2-2 |
| One or more antiperspirant or | 0-30 | 10-25 | 15-20 |
| odor supressing metal | | | |
| compounds | | | |
| One or more solid fillers | 0-10 | 1.5-8 | 2-5 |

The topical aerosol deodorant base composition summarized in the above table may be further comprise one or more antioxidants agents, pH adjusting agents, buffering agents, chelating agents and combinations of the foregoing. The topical aerosol base composition should have a target pH of 3.5 to 3.9; a target viscosity of 0 to 500 cps at 25° C. and a target shelf life of 1-2 years.

The topical aerosol deodorant base composition summarized in the above table is filled into a suitable aluminum container along with a propellant and sealed. The propellant is preferably an inert gas, most preferably nitrogen in amount of 1% or less.

### Example 2

A topical aerosol deodorant base composition, i.e. without the propellant, in accordance with the present invention may be prepared having the following:

| Ingredient | Weight Percent |
|---|---|
| Water | 50-70 |
| Denatured Ethanol (SD Alcohol 40B) | 10-30 |
| Mandelic Acid | 1-6 |
| Caffeine | 0.5-5 |
| Caprylic/Capric Triglyceride | 1-10 |
| Oleyl Alcohol | 0.5-5 |
| Coco-Caprylate | 1-8 |
| Undecane | 1-10 |
| Tridecane | 1-10 |

The topical aerosol deodorant base composition summarized in the above table may be further comprise one or more fragrances, including but not limited to a citronella compound such as citronellyl methycrotonate or citronellyl acetate, one or more preservatives, such as benzyl alcohol, benzoic acid, sorbic acid or combinations thereof, antioxidants agents such as citric acid, pH adjusting agents, buffering agents, chelating agents and combinations of the foregoing. These additional additives may be added to the base composition prior to adding the base composition to the dispensing container, concurrently with the addition of the base composition to the dispensing container or subsequent to or after the base composition has been added to the dispensing container. The topical aerosol base composition should have a target pH of 3.5 to 3.9; a target viscosity of 0 to 500 cps at 25° C. and a target shelf life of 1-2 years.

The topical aerosol deodorant base composition summarized in the above table, with or without the additional additives is filled into a suitable aluminum dispensing container along with a propellant and sealed. The propellant is preferably an inert gas, most preferably nitrogen in amount of 1% or less.

The invention described herein may be practiced in the absence of any element or limitation, which is not specifically disclosed herein. Thus, for example, in each instance herein, any of the terms “comprising,” “consisting essentially of” and “consisting of” may be replaced with either of the other two terms. The terms and expressions, which have been employed, are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A topical aerosol deodorant comprising:
A) a base composition comprising:
(i) about 40 wt % to about 80 wt % water;
(ii) about 5 wt % to about 50 wt % of one or more organic solvents;
(iii) about 1 wt % to about 10 wt % of one or more alpha-hydroxycarboxylic acids;
(iv) about 1 wt % to about 40 wt % of one or more skin conditioning agents;
(v) about 1 wt % to about 30 wt % of one or more hydrocarbon carrier agents;
(vi) 0 wt % to about 10 wt % of caffeine or a xanthine derivative that has vasoconstrictive effects; and
(vii) optionally one or more additional cosmetically acceptable auxiliary substances selected from the group consisting of preservative agents, antioxidants agents, pH adjusting agents, buffering agents, chelating agents, fragrance agents and combinations of the foregoing and
B) a propellant.

2. The topical aerosol deodorant composition of claim 1 wherein the one or more organic solvents comprises a C₁ to C₆ alcohol.

3. The topical aerosol deodorant composition of claim 1 wherein the one or more alpha-hydroxycarboxylic acids is selected form the group consisting of mandelic acid, lactic acid and combinations thereof.

4. The topical aerosol composition of claim 1 wherein the one or more skin conditioning agents are a liquid at room temperature and are selected from mineral oils, vegetable oils, triglycerides, fatty acids, fatty alcohols, fatty acid esters and combinations thereof.

5. The topical aerosol deodorant composition of claim 1 wherein the one or more hydrocarbon carrier agents are selected from the group consisting of undecane, tridecane and combinations thereof.

6. The topical aerosol deodorant composition of claim 1 wherein the propellant is a compressed gas selected from the group consisting of compressed air, nitrogen, inert Nobel gas, carbon dioxide, nitrous oxide, and combinations thereof.

7. The topical aerosol deodorant composition of claim 1 wherein the propellant comprises nitrogen.

8. The topical aerosol deodorant composition of claim 1 that is free of solid fillers.

9. The topical aerosol deodorant composition of claim 1 that is free of metal compounds.

10. The topical aerosol deodorant composition of claim 1 that is free of parabens.

11. The topical aerosol deodorant composition of claim 1 that is free of phthalates.

12. The topical aerosol deodorant composition of claim 1 that is free of sulfites.

13. The topical aerosol deodorant composition of claim 1 that is free of polymeric film forming agents.

14. A topical aerosol deodorant comprising:
A) a base composition comprising:
(i) about 40 wt % to about 80 wt % water;
(ii) about 5 wt % to about 50 wt % of one or more C₁ to C₆ alcohols;
(iii) about 1 wt % to about 10 wt % of one or more alpha-hydroxycarboxylic acids selected form the group consisting of mandelic acid, lactic acid and combinations thereof;
(iv) about 1 wt % to about 40 wt % of one or more skin conditioning agents that are a liquid at room temperature and selected from the group consisting of mineral oils, vegetable oils, triglycerides, fatty acids, fatty alcohols, fatty acid esters and combinations thereof;
(v) about 1 wt % to about 30 wt % of one or more hydrocarbon carrier agents selected from the group consisting of undecane, tridecane and combinations thereof;
(vi) 0 wt % to about 10 wt % of caffeine; and
(vii) one or more additional cosmetically acceptable auxiliary substances selected from the group consisting of preservative agents, antioxidants agents, pH adjusting agents, buffering agents, chelating agents, fragrance agents and combinations of the foregoing and
B) a nitrogen propellant.

15. The topical aerosol composition of claim 14 wherein the one or more skin conditioning agents comprise caprylic/capric triglycide, oleyl alcohol, coco-caprylate and combinations thereof.

16. The topical aerosol composition of claim 14 further comprising one or more citronella fragrance agents.

17. The topical aerosol deodorant composition of claim 14 that is free of solid fillers, free of metal compounds, free of parabens, free of phthalates, free of sulfites and free of polymeric film forming agents.
